(19) United States
(12) Patent Application Publication
(10) Pub. No.: US 2010/0059120 A1
(43) Pub. Date: Mar. 11, 2010

(54) MICROFLUIDIC DEVICE AND METHODS FOR DROPLET GENERATION AND MANIPULATION

(75) Inventors: Wei-Cheng Tian, Clifton Park, NY (US); Jeffrey Bernard Fortin, Chelmsford, MA (US); Jun Xie, Niskayuna, NY (US); Barbara Grossman, Clifton Park, NY (US); Oliver Charles Boomhower, Waterford, NY (US); Shashi Thutupalli, Bangalore (IN); Long Que, Rexford, NY (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/208,445

(22) Filed: Sep. 11, 2008

(51) Int. Cl. G01N 33/00

(52) U.S. Cl. 137/1; 422/68.1

(57) ABSTRACT

Methods and microfluidic devices for generating and manipulating sample droplets, wherein the devices comprise, a plurality of fluid channels, at least one of which is a sample channel for carrying a fluidic sample material, that is in fluid communication with the carrier fluid channel via an orifice; and an actuated flow interrupter adapted to force a predetermined amount of the sample fluid from the sample channel through the orifice into the carrier fluid channel.

### BACKGROUND

The invention relates generally to microfluidic devices and the need to generate nanometer/micrometer size droplets of fluid within these devices. Such devices are useful in a wide variety of applications including bio- and biochemical assays, multiplexing, high content analysis chips and cell sorting systems.

As shown in FIG. 1, previous approaches to forming droplets typically combine the flow rates of two fluids within the microchannels and the shear forces acting between these two fluids, to effectively shear off droplets of fluid. These two fluids are necessarily located in the same plane using such an approach. As the carrier fluid, such as oil, flows along the microchannel, the protein solution, buffer and a salt solution are introduced together into the microchannel to form a droplet carried along by the carrier fluid. The flow rate of the carrier fluid and formation of the droplets are determined by dynamic metering or flow rate of these two fluids. The protein solution, buffer and a salt solution are rapidly mixed together within the droplet by chaotic advection whereby microchannel comprises a series of turns. FIG. 1 shows one cycle of advection. However, when using this approach, the size of the droplets is inconsistent from one droplet to another and as a result it is difficult to accurately quantify the ratio of materials in each sample droplet.

### BRIEF DESCRIPTION

Unlike the existing approaches to droplet formation, the methods and devices of the invention, use an on-chip and/or off-chip actuation to induce the shear force needed to introduce droplets into a carrier fluid. The formation of the droplets is easier to control which results in droplets having a consistent size and composition.

The methods and microfluidic devices may be used for a variety of applications such as, but not limited to, analysis, separation and culturing of biomaterials, such as protein analysis, protein separation, nucleic acid and protein amplification, and on-chip cell culturing.

An example embodiment of the microfluidic device of the invention for generating and manipulating sample droplets, generally comprises, at least one carrier fluid channel; at least one sample channel for carrying a fluidic sample material, that is in fluid communication with the carrier fluid channel via an orifice; an actuated flow interrupter adapted to force a predetermined amount of the sample fluid from the sample channel through the orifice into the carrier fluid channel.

The flow interrupter may comprise, for example, a deformable membrane provided along a wall of the sample channel across from, or proximal to, the orifice; and a membrane deforming actuator. The actuator may comprise, but is not limited to, one, or a combination, of: a pneumatic controller, an electrostatic drive, two opposing electrodes, a magnetic component, or a piezoelectric component.

The flow interrupter may also, or alternatively comprise a deformable sphere provided integrated into a wall of the sample channel across from, or proximal to, the orifice; and a sphere expansion actuator, wherein the sphere expansion actuator may comprise, but is not limited to, a temperature or pressure control.

Another example embodiment of the microfluidic device of the invention, for generating and manipulating sample droplets, generally comprises, a first fluid channel; a second fluid channel, that is in fluid communication with the first fluid channel via an orifice; a deformable membrane provided along a wall of the second channel across from, or proximal to, the orifice, and adapted to force a predetermined amount of the sample fluid from the sample channel through the orifice into the carrier fluid channel; and a membrane deforming actuator.

The actuator may comprise, but is not limited to, one or combination of, a pneumatic controller, an electrostatic drive, two opposing electrodes, a magnetic component or a piezoelectric component.

An example of the methods of the invention for generating and manipulating microfluidic droplets, generally comprises the steps of, introducing a carrier fluid into a first microfluidic channel; introducing a fluidic sample into a second microfluidic channel, that is in fluid communication with the first microfluidic channel via an orifice; and initiating an actuated flow interrupter to force a predetermined amount of the fluidic sample from the sample channel through the orifice into a carrier fluid stream in the first channel whereby the fluidic sample droplet retains its droplet characteristic in the carrier fluid stream. The fluidic sample may comprise one or more biomaterials.

In at least one of the examples of the methods, the flow interrupter comprises a deformable membrane provided along a wall of the sample channel across from, or proximal to, the orifice; whereby the membrane is deformed by a membrane deforming actuator, causing the predetermined amount of the fluidic sample to shear off from the fluidic sample and move through the orifice into the carrier fluid stream in the first channel.

### DRAWINGS

These and other features, aspects, and advantages of the present invention will become better understood when the following detailed description is read with reference to the accompanying drawings in which like characters represent like parts throughout the drawings, wherein:

FIG. 1 is a schematic drawing of a previous approach to droplet formation in which the shear forces of the flow rates, between the carrier fluid and the sample stream being introduced into the flowing carrier fluid, determine the size and compositional ratio of the droplets.

FIG. 2 is a cross-sectional side view of an embodiment of the droplet formation device of the invention.

FIG. 3A is a top view of the embodiment shown in FIG. 2.

FIG. 3B is a bottom view of the embodiment shown in FIG. 2.

FIG. 4 is an enlarged schematic view of the formation of a droplet using an embodiment of the droplet formation device of the invention.

FIG. 5 is a schematic drawing of an embodiment of an out-of-plane pneumatic actuator of the device of the invention.

FIG. 6 is a schematic drawing of an embodiment of an in-plane electrostatic actuator of the device of the invention.

FIG. 7 is a schematic drawing of an embodiment of an out-of-plane electrostatic actuator of the device of the invention.

FIG. 8 is a schematic drawing of an embodiment of an out-of-plane piezoelectric actuator of the device of the invention.

FIG. 9 is a schematic drawing of an embodiment of an in-plane thermal actuator of the device of the invention.

FIG. 10 is a schematic drawing of an embodiment of an out-of-plane thermal actuator of the device of the invention.

FIG. 11 is a schematic drawing of an embodiment of sphere actuator of the device of the invention.

FIG. 12 is an enlarged schematic drawing of the expansion and contraction of the flow-interrupting sphere shown in FIG. 11.

FIG. 13 is a graph of the maximum displacement relative to width of an embodiment of a flow-interrupting membrane of the device of the invention.

FIG. 14 is a graph of the maximum displacement relative to the force applied to an embodiment of a flow-interrupting membrane of the device of the invention.

FIG. 15A is a top view of a microfluidic chip, in which four of the microfluidic devices of the invention are shown.

FIG. 15B is an enlarged top view of one of the microfluidic devices shown in FIG. 15A.

FIG. 16 is a cross-sectional view of an embodiment of the microfluidic device of the invention.

FIG. 17 is an exploded cross-sectional side view of an example of a layering process that may be used for fabricating the microfluidic device of the invention.

FIG. 18 is a schematic side view, perpendicular to the view of FIG. 16 in the same plane, illustrating an example of a surface machining process that may be used for fabricating the microfluidic device of the invention.

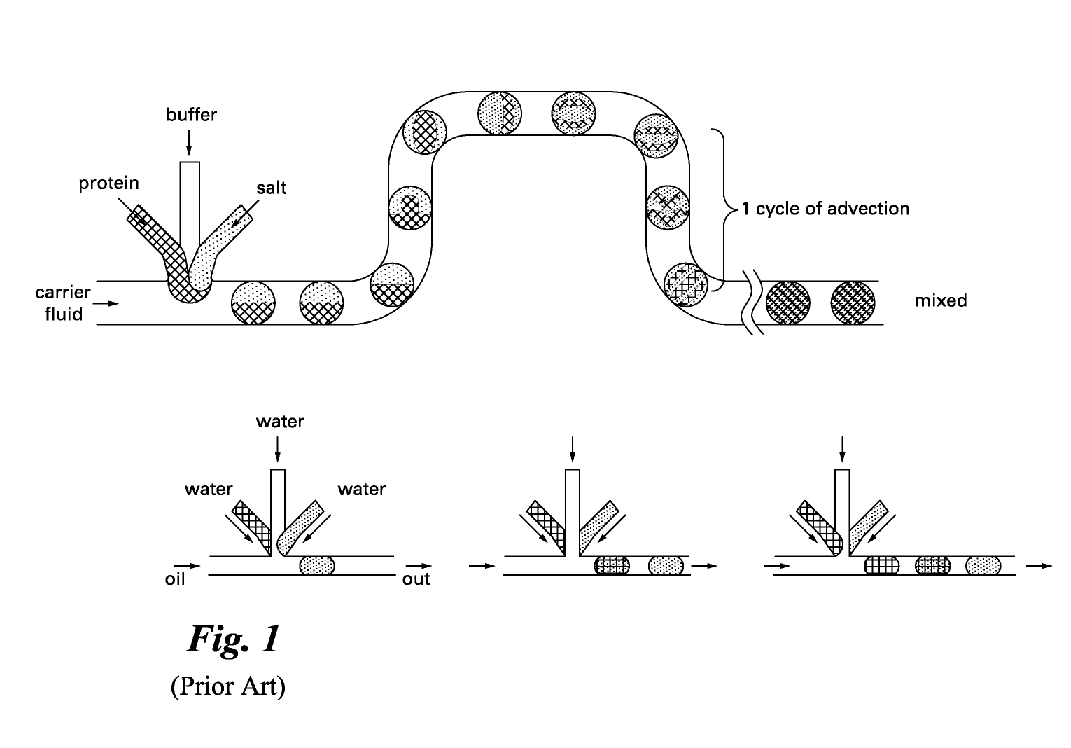

### DETAILED DESCRIPTION

To more clearly and concisely describe and point out the subject matter of the claimed invention, the following definitions are provided for specific terms that are used in the following description.

As used herein, the term “biomaterial” refers to material that is, or is obtained from, a biological source. Biological sources include, for example, biological and biochemical materials derived from, but are not limited to, bodily fluids (e.g., blood, blood plasma, serum, or urine), organs, tissues, fractions, cells, cellular, subcellular and nuclear materials that are, or are isolated from, single-cell or multi-cell organisms, fungi, plants, and animals such as, but not limited to, insects and mammals including humans. Biological sources include, as further nonlimiting examples, materials used in, or derived from, monoclonal antibody production, GMP inoculum propagation, insect cell cultivation, stem cell propagation and differentiation, gene therapy, perfusion, E. coli propagation, protein expression, protein amplification, plant cell culture, pathogen propagation, cell therapy, bacterial production and adenovirus production.

As used herein, the term “carrier fluid” refers to any fluid, without limitation on the density, viscosity or chemical or biological composition of the fluid, in which particulates are suspended or, otherwise, carried and is not limited to any specific composition or material. The term is used only to distinguish the carrier fluid from the droplet materials or fluidic samples, particles or particulate matter for purposes of this description. The terms droplet materials, fluidic samples, particles and particulate matter are used interchangeably and are not limiting, and include any particle or matter that can be suspended, at least temporarily, in a carrier fluid.

The devices generally comprise a microfluidic device to generate and manipulate sample droplets along one or more microfluidic channels. In one example embodiment, the device comprises at least two microchannels, one carrying the carrier fluid and the other the sample fluid materials, connected, or otherwise in fluid communication with each other, at least in part, via an orifice. One or more of the example embodiments also further comprise a flow interrupter that momentary interrupts the flow of sample fluid in the sample fluid channel and forces a discrete amount of sample fluid through the orifice into the flow stream of the carrier fluid in the other channel. One example embodiment of the flow interrupter is a deformable membrane provided along a wall of the sample fluid channel across from, or in close proximity to, the orifice. The flow interrupter, e.g. a membrane, may be actuated using various types of actuators.

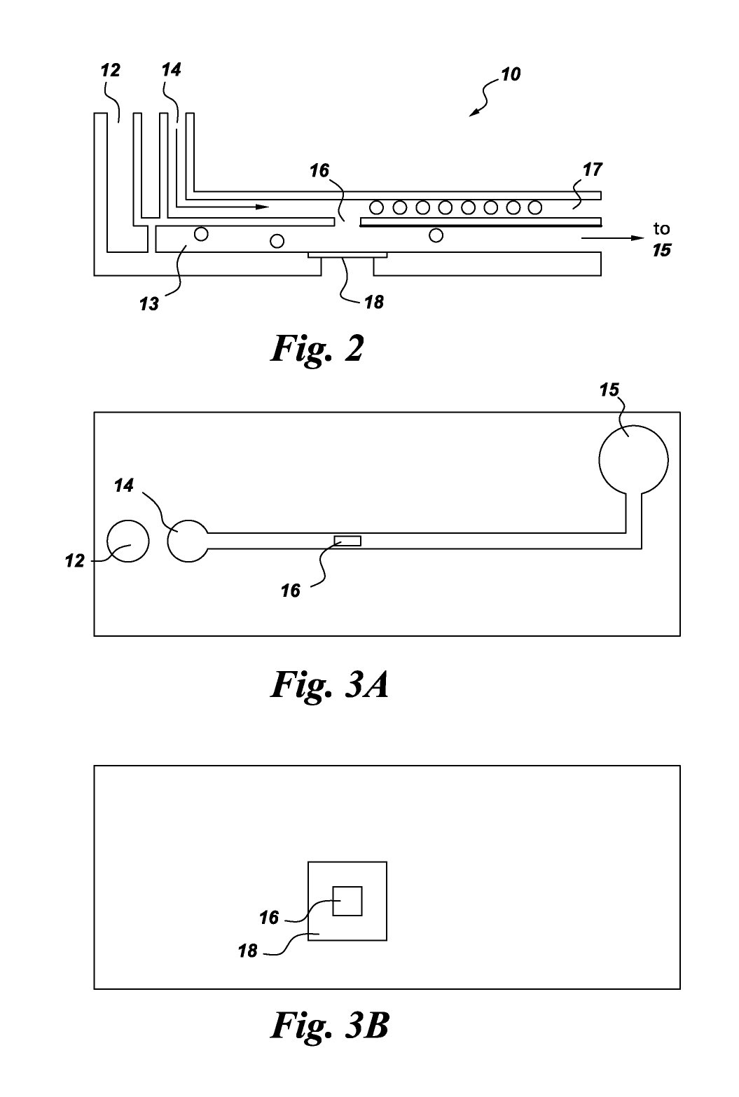

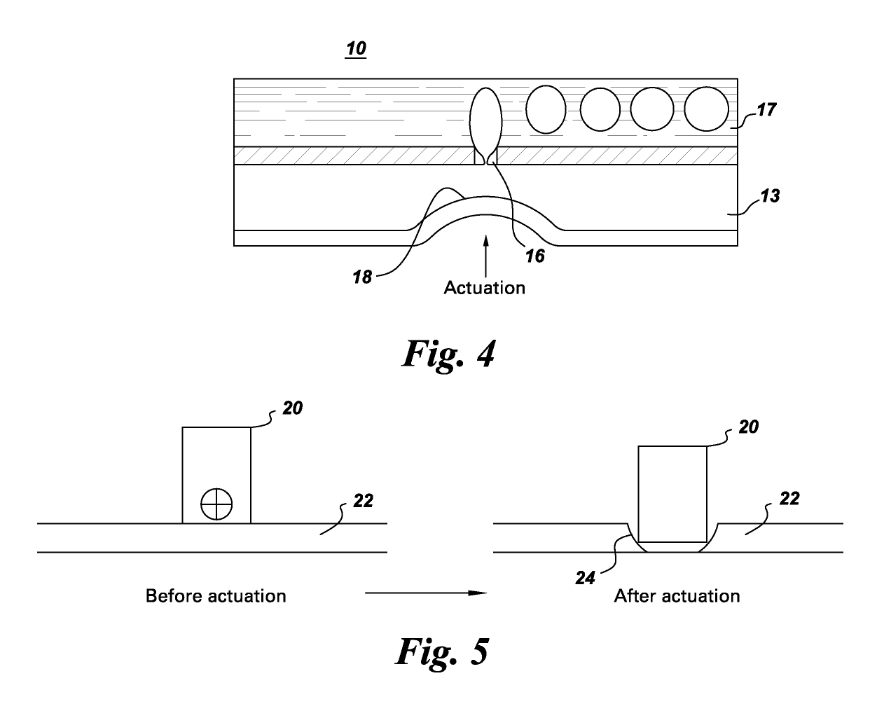

An example embodiment of the droplet formation device is shown and generally referred to in FIGS. 2, 3A and 3B, as device. Device 10 comprises input channel 12 and input channel 14 into which the sample fluid is loaded into channel 13 and the carrier fluid into channel 17. As the fluids flow past orifice 16, membrane 18 is deformed in the direction of arrow A to momentarily impede the flow of the sample fluid in channel 13, forcing a discrete amount of sample fluid through orifice 16 into channel 17. Each droplet forced through orifice 16 enters the flow steam of the carrier fluid and continues along channel 17 for any number of purposes, such as cell sorting, sample detection, or culturing. Excess sample material not forced through orifice 16 continues along channel 13 to one or more reservoirs 15. FIG. 4 shows an enlarged view of the formation of droplets in device 10. In one or more of the embodiments, the inner surfaces of channels 12, 13, 14 and 17 are hydrophobic to maintain the flow rate of the carrier fluid and sample fluids along their respective channel and to ensure smooth droplet formation through orifice 16.

Membrane 18 can be deformed using an on-chip or off-chip actuator. Although various methods and devices may be used as the actuator such as but not limited to, pneumatic, electrostatic, piezoelectric, magnetic and hydraulic, a few example embodiments are provided. FIG. 5 illustrates an example embodiment of a pneumatic actuator 20 before actuation and after actuation. As the sample fluid flows through channel 22, air is forced into actuator 20 causing it to push against and deform membrane 24 into the sample stream in channel 22.

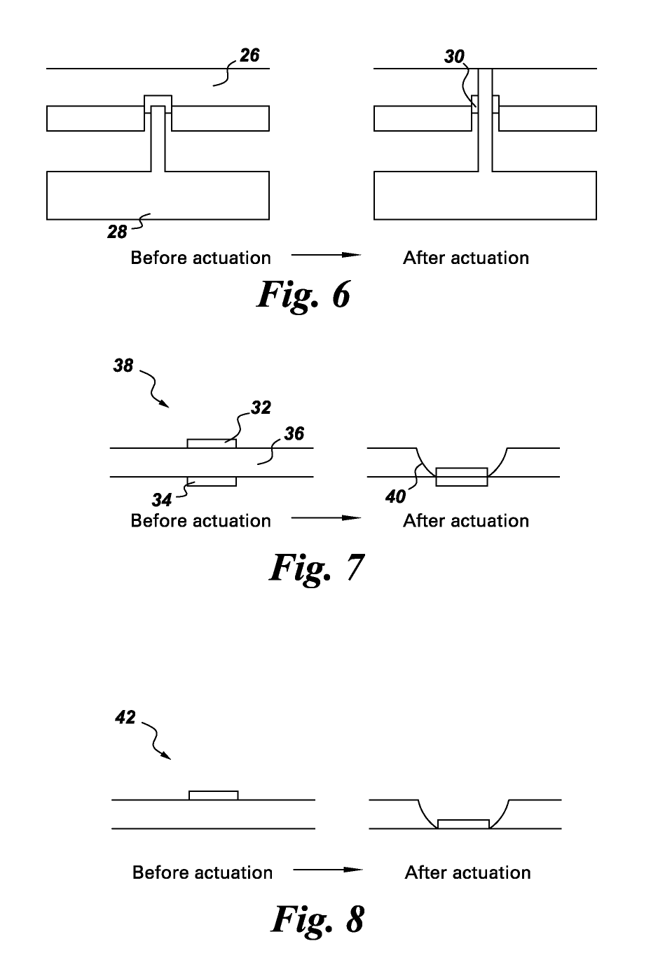

FIGS. 6 and 7 illustrate two example embodiments of electrostatic actuators. The electrostatic actuator shown in FIG. 6 is an example of an in-plane actuator. As the sample fluid flows through channel 26, the electrostatic pressure forces actuator 28 into the sample stream to impede the flow and forcing a droplet through orifice 30. The electrostatic actuator shown in FIG. 7 is an example of an out-of-plane actuator comprising electrodes 32 and 34 located on opposite sides of sample fluid channel 36. An electrostatic charge causes actuator 38 to push against membrane 40 and impede the flow of the sample fluid in channel 36. An example embodiment of a piezoelectric actuator 42 is illustrator in FIG. 8.

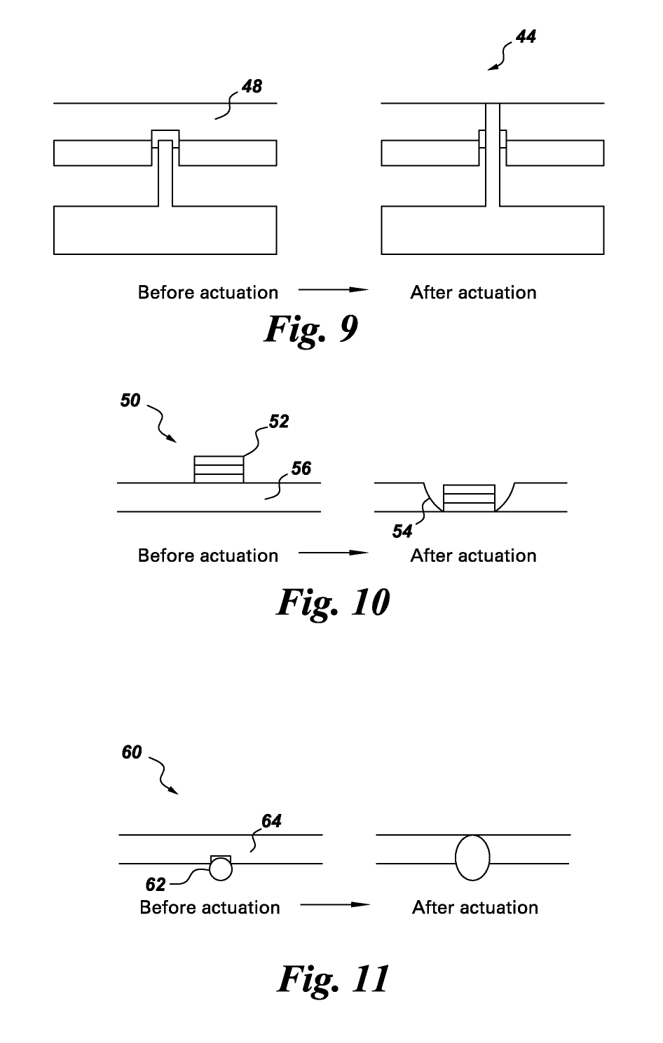

FIGS. 9 and 10 illustrate two example embodiments of thermal-based actuators. FIG. 9 shows an example of an in-plane thermal actuator and FIG. 10 shows an out-of-plane thermal actuator. Thermal actuator 44 shown in FIG. 9 comprises a passive valve, which prevents the sample fluid from leaking into the actuator, and a bent-beam that, when actuated, is pushed into channel 48 to interrupt the flow of sample fluid. Thermal actuator 50 shown in FIG. 10 comprises an integrated bimorph actuator 52 that, when triggered, pushes against membrane 54 to interrupt the flow of sample fluid in channel 56.

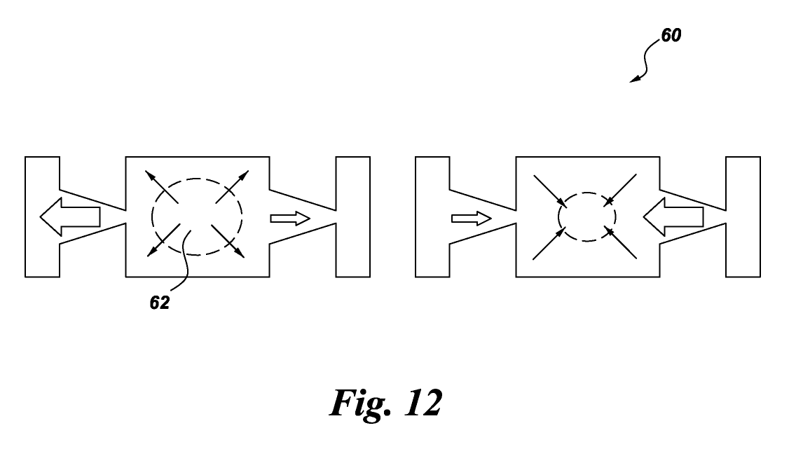

FIGS. 11 and 12 illustrate an example embodiment of an actuator that comprises a balloon or bubble that expands and contracts to momentarily interrupt the flow of sample fluid. Actuator device 60 comprises a hollow deformable sphere 62 that expands and contracts, a shown in a top view in FIG. 12, into sample fluid channel 64 to interrupt the flow of sample fluid. The expansion and contraction of sphere 62 can be achieved by heating and cooling a material in the sphere, such as a gas or liquid. The sphere itself may made of a thermally expandable material capable of expanding and contracting by heating and cooling without the need for a separate gas or liquid.

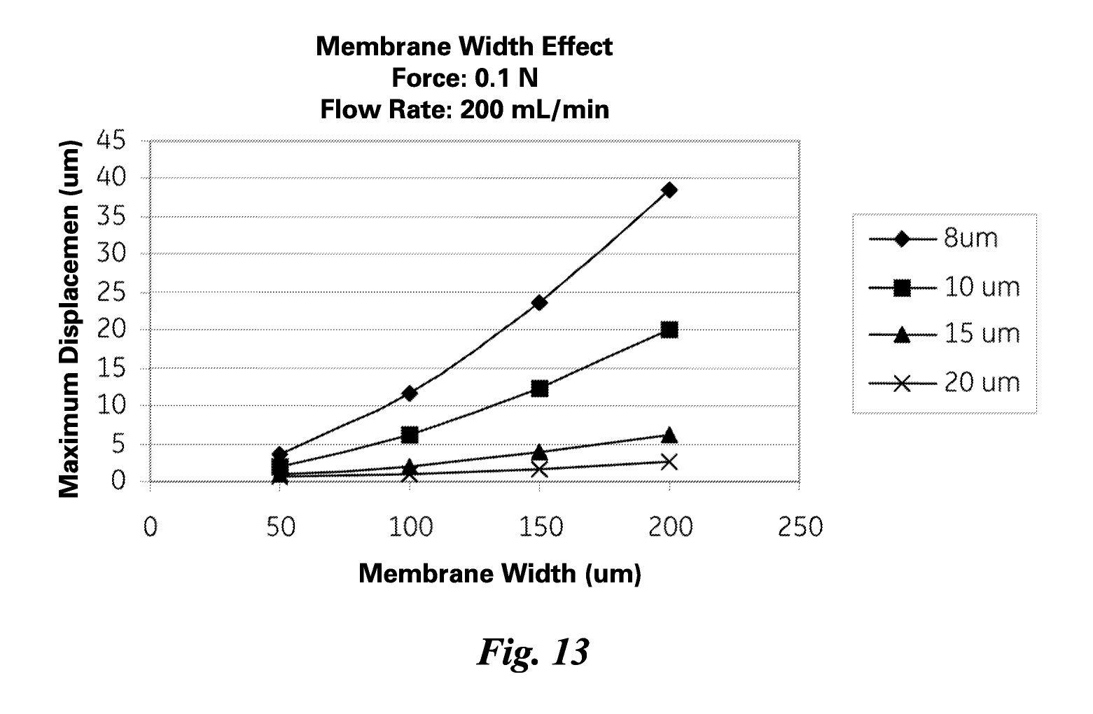

A graph of the membrane width versus the maximum displacement is shown in FIG. 13. The data is shown for four example membranes, having a thickness of 8, 10, 15 and 20 um, respectively, in which the force is 0.1 N and the flow rate is 20 nL/min. In this example, the width of the sample fluid channel is 50 um and the channel depth is 50 um. As the graph shows, for example, a membrane having a thickness of 8 um and a width of 100 um will be displaced about 12 um when subjected to a force of 0.1 N. Example ranges of membrane thickness and width include, but are not limited to, a membrane thickness between about 0.1 um to 100 um and a membrane width between about 5 um to 5000 um. One specific, although non-limiting example, has a membrane thickness between about 8 um to 20 um and a membrane width between 50 um and 500 um. Actuator should be configured to apply a force suited to the thickness and width of the membrane. For example, a system controller may be configured to, but is not limited to, apply an actuated force on the membrane between 0.0001 N to 1.0 N. One specific, although non-limiting example, is adapted to apply a force on the membrane between 0.01 N to 0.1 N. The system may be programmed to apply a predetermined force or may comprise a sensing subsystem capable of adjusting the applied force based on operating conditions and the thickness and width of the membrane.

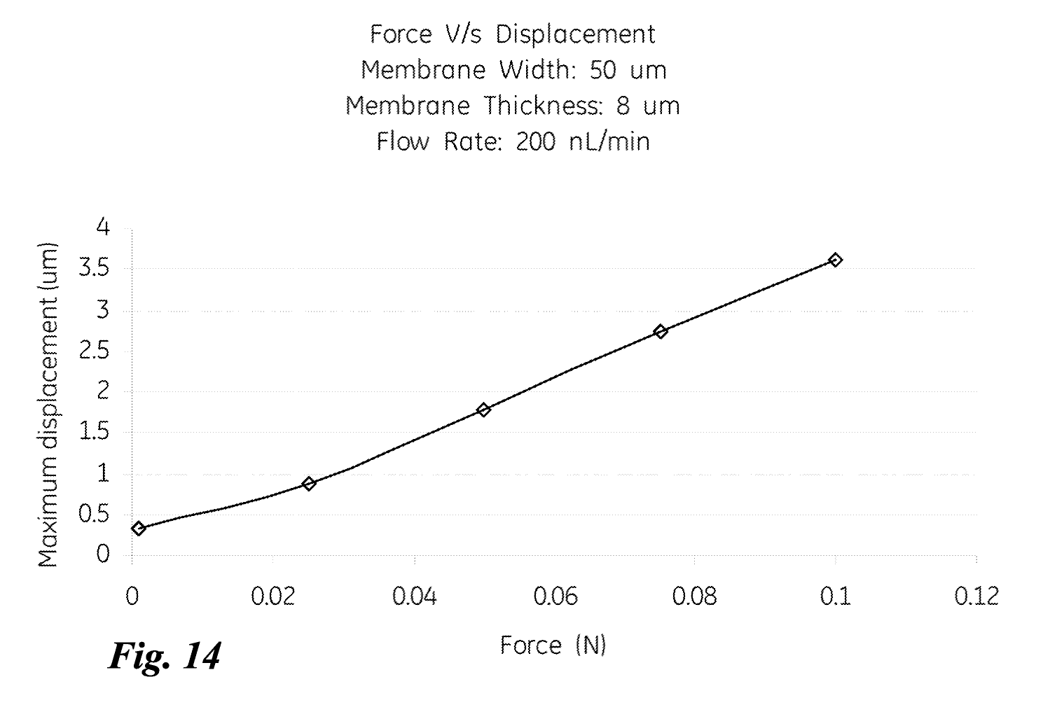

FIG. 14 shows a graph of the maximum displacement of a membrane that is 50 um wide and 8 um thick when subjected to increasing levels of force (N). The flow rate in this example is 200 nL/min. As the graph shows, in this example, the displacement of the membrane increases from about 0.7 um to about 3.6 um when the force increases from about 0.02 N to about 0.1 N.

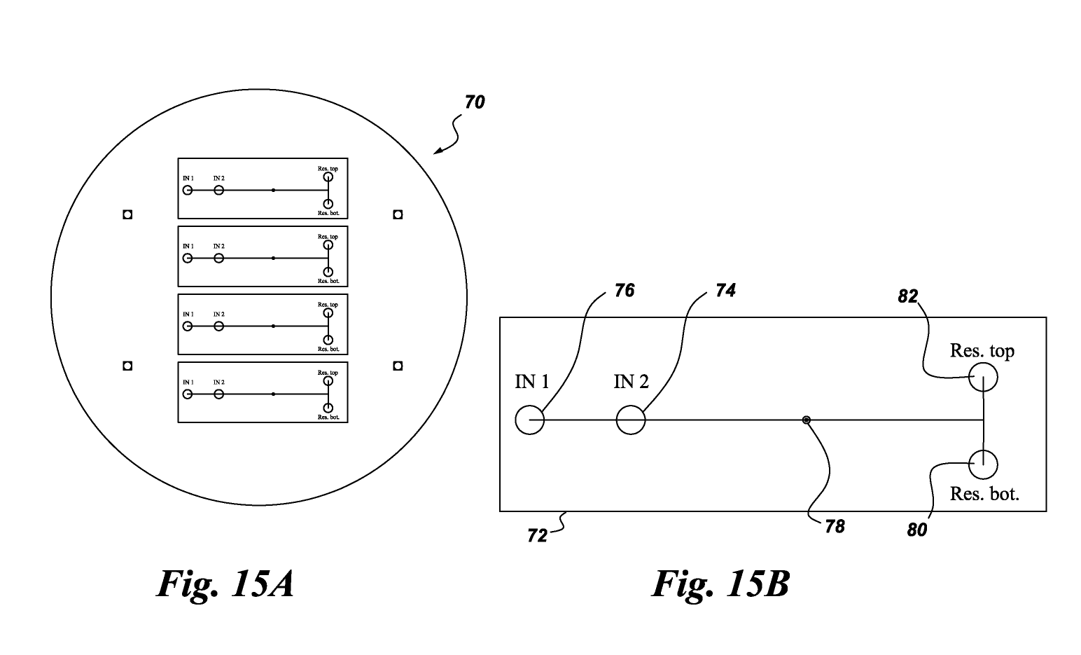

FIGS. 15A and 15B show a top view of an embodiment of a microfluidic chip 70 comprising four channel devices and an enlarged view of one of those four channel devices 72, respectively. Device 72 comprises input well 74 into which the carrier fluid is introduced and input well 76 into which the sample fluid is introduced. Orifice 78 is located to the right of the input wells in the direction of the fluid flow. Although the orifice may be any size and shape suited to forming a given droplet, examples of orifice diameters include, but are not limited to, a range between 5 to 1500 um. More specific, although non-limiting, examples of orifice diameters are 50, 80, 100 and 150 um. Reservoirs 80 and 82, for the access sample fluid and the waste fluid are located at the distal end of the channels in the direction of the fluid flow.

Below is a table of nonlimiting parameters for one example embodiment.

| Major | Channel | Channel | Membrane | Orifice size |
|---|---|---|---|---|
| parameters | width (um) | depth (um) | size (um) | (um) |
| top | 50 | 50 | | 50, 80, 100, |
| | | | | 150 |
| bottom | 50 | 50 | 500 (8 um | |
| | | | thick) | |

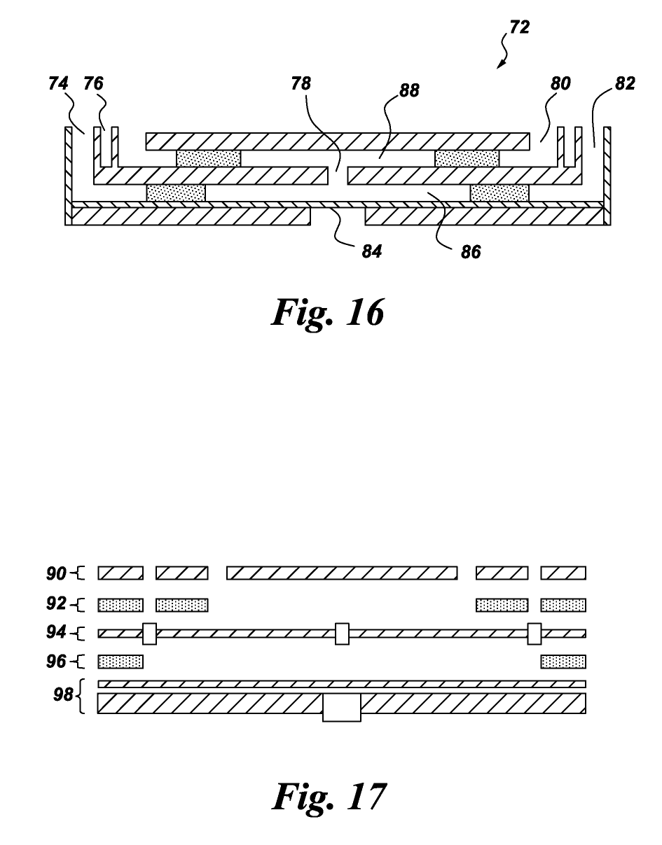

FIG. 16 is a cross-sectional view of microfluidic device 72 showing input well 74 into which the sample is introduced and input well 76 into which the carrier fluid in introduced. Orifice 78 is located to the right of the input wells in the direction of the fluid flow. Reservoirs 80 and 82, for the access carrier fluid and the waste fluid are located at the distal end of the channels in the direction of the fluid flow. Membrane 84 is located on the underside of sample fluid channel 86 and below orifice 78 located on the upper side of carrier channel 86 and the underside of carrier fluid channel 88.

FIG. 17 is an exploded cross-sectional side view of the layering process for fabricating microfluidic device 72 using a Kapton fabrication process to form the channel layers, input wells, reservoirs, membrane and orifice.

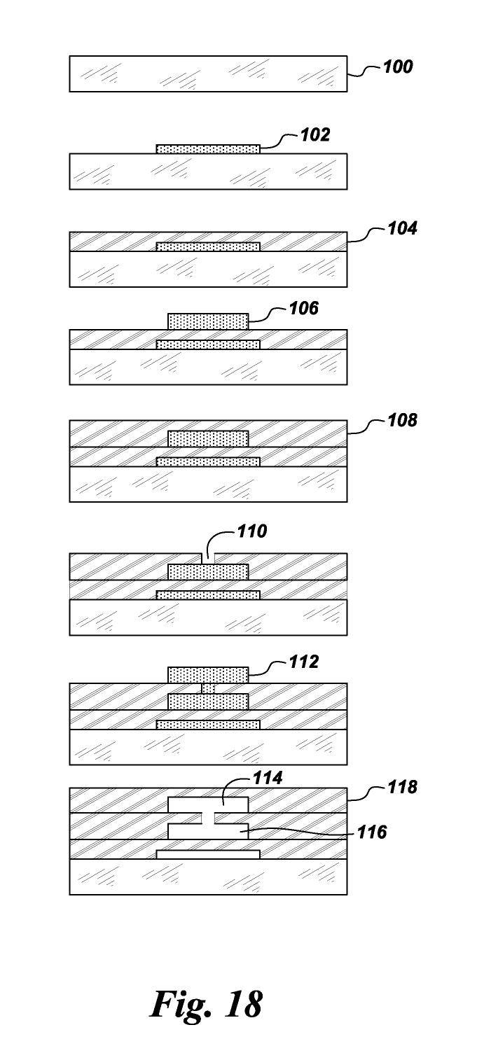

FIG. 18 is a schematic side view, perpendicular to the view of FIG. 16 in the same plane, illustrating a surface machining process for fabricating microfluidic device 72, beginning with a suitable glass substrate. A sacrificial layer is applied followed by an integrated actuator membrane layer comprising parylene, PDMS or any other suitable polymer. Another sacrificial layer is applied, which will form the sample fluid channel, followed by another polymer layer, through which the orifice is etched. A third sacrificial layer is applied, which will form the carrier fluid channel, which is then followed by another polymer layer which forms the top cover of the device.

The microfluidic devices and methods are adaptable for a variety of uses including, but not limited to, cell sorting, high throughput drug screening, biological analysis, chemical analysis, biological separation, chemical separation, cell culturing, biological amplification or chemical amplification.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A microfluidic device for generating and manipulating sample droplets, comprising,
at least one carrier fluid channel;
at least one sample channel for carrying a fluidic sample material, that is in fluid communication with the carrier fluid channel via an orifice; and
an actuated flow interrupter adapted to force a predetermined amount of the sample fluid from the sample channel through the orifice into the carrier fluid channel.

2. The microfluidic device of claim 1, wherein the flow interrupter comprises a deformable membrane provided along a wall of the sample channel across from, or proximal to, the orifice; and a membrane deforming actuator.

3. The microfluidic device of claim 2, wherein the actuator comprises a pneumatic controller.

4. The microfluidic device of claim 2, wherein the actuator comprises an electrostatic drive.

5. The microfluidic device of claim 2, wherein the actuator comprises two opposing electrodes.

6. The microfluidic device of claim 2, wherein the actuator comprises at least one magnetic component.

7. The microfluidic device of claim 2, wherein the actuator comprises a piezoelectric component.

8. The microfluidic device of claim 1, wherein the flow interrupter comprises a deformable sphere provided integrated into a wall of the sample channel across from, or proximal to, the orifice; and a sphere expansion actuator.

9. The microfluidic device of claim 8, wherein the sphere expansion actuator comprises a temperature or pressure control.

10. The microfluidic device of claim 2, wherein the membrane has a thickness between about 0.1 um to 100 um and a width between 5 um and 5000 um, and the actuator is adapted to apply a force on the membrane between 0.0001 N to 1.0 N.

11. The microfluidic device of claim 2, wherein the membrane is about 8 um thick and about 50 um wide and the actuator is adapted to apply a force on the membrane between 0.01 N to 0.1 N.

12. The microfluidic device of claim 11, wherein the orifice has an opening width between about 5 and 1500 um.

13. The microfluidic device of claim 1, wherein the membrane comprises Kapton.

14. The microfluidic device of claim 2, wherein the membrane has a thickness between about 8 um to 20 um and a width between about 50 um to 500 um.

15. The microfluidic device of claim 2, wherein the actuator is adapted to apply a force on the membrane between 0.01 N to 0.1 N.

16. A microfluidic device for generating and manipulating sample droplets, comprising,
a first fluid channel;
a second fluid channel, that is in fluid communication with the first fluid channel via an orifice;
a deformable membrane provided along a wall of the second channel across from, or proximal to, the orifice, and adapted to force a predetermined amount of the sample fluid from the sample channel through the orifice into the carrier fluid channel; and
a membrane deforming actuator.

17. The microfluidic device of claim 16, wherein the first fluid channel comprises a cell sorter.

18. The microfluidic device of claim 16, wherein the first fluid channel comprises an imaging zone.

19. The microfluidic device of claim 16, wherein the inner surfaces of the first and second fluid channels and the orifice are hydrophobic.

20. The microfluidic device of claim 16, wherein the actuator comprising one or a combination of, a pneumatic controller, an electrostatic drive, two opposing electrodes, a magnetic component or a piezoelectric component.

21. A method for generating and manipulating microfluidic droplets, comprising the steps of,
introducing a carrier fluid into a first microfluidic channel;
introducing a fluidic sample into a second microfluidic channel, that is in fluid communication with the first microfluidic channel via an orifice; and
initiating an actuated flow interrupter to force a predetermined amount of the fluidic sample from the sample channel through the orifice into a carrier fluid stream in the first channel whereby the fluidic sample droplet retains its droplet characteristic in the carrier fluid stream.

22. The method of claim 21, wherein the flow interrupter comprises a deformable membrane provided along a wall of the sample channel across from, or proximal to, the orifice; and whereby the membrane is deformed by a membrane deforming actuator, causing the predetermined amount of the fluidic sample to shear off from the fluidic sample and move through the orifice into the carrier fluid stream in the first channel.

23. The method of claim 22, wherein the fluidic sample comprises one or more biomaterials.

24. The method of claim 21, wherein the microfluidic channels are housed in a microfluidic device that is adapted for use in cell sorting, high throughput drug screening, biological analysis, chemical analysis, biological separation, chemical separation, cell culturing, biological amplification or chemical amplification.

25. A microfluidic device for generating and manipulating sample droplets, comprising,
at least one carrier fluid channel;
at least one sample channel for carrying a fluidic sample material, that is in fluid communication with the carrier fluid channel via an orifice; and
an actuated flow interrupter adapted to force a predetermined amount of the sample fluid from the sample channel through the orifice into the carrier fluid channel; and
wherein the sample channel is adapted for use adapted for use in cell sorting, high throughput drug screening, biological analysis, chemical analysis, biological separation, chemical separation, cell culturing, biological amplification or chemical amplification
